(19) United States
(12) Patent Application Publication
(10) Pub. No.: US 2016/0143918 A1
(43) Pub. Date: May 26, 2016

(54) DRUG RELEASING AGENT BASED ON beta-SITOSTEROL AND A PREPARATION METHOD THEREOF

(71) Applicant: School of Medicine Jiaying University, Meizhou (CN)

(72) Inventor: Jinsheng Cheng, Meizhou (CN)

(21) Appl. No.: 14/948,250

(22) Filed: Nov. 21, 2015

(30) Foreign Application Priority Data
Nov. 26, 2014 (CN) 201410689842.2

(51) Int. Cl. A61K 31/575; A61K 9/20; A61K 47/48

(52) U.S. Cl. CPC A61K 31/575; A61K 47/48969; A61K 9/2095; A61K 9/205

(57) ABSTRACT

The present invention relates to the technical field of β-sitosterol drugs and provides a drug sustained release agent based on β-sitosterol and a preparation method thereof. The drug sustained release agent based on the β-sitosterol is applied to drugs with the β-sitosterol as a main drug component and is prepared from the components including a drug carrier, a hydrophilic gel material, a erodible matrix material and an insoluble matrix material, wherein the drug carrier is a β-cyclodextrin-polyamide-amine dendrimer composites, β-sitosterol is from a plant raw material, and a host-guest inclusion complex is composed of the main drug component and the drug carrier according to the mass ratio of 0.1:0.1-0.1:5. A preparation method comprises the following steps: preparing the inclusion complex, mixing auxiliaries, carrying out compression moulding and the like. The drug sustained release agent based on β-sitosterol has the characteristics of stable drug concentration, high biological activity, good drug solubility and long acting effect.

### CROSS-REFERENCE TO RELATED APPLICATIONS

This application is a continuation in part of, and claims priority to, Chinese Patent Application No. 201410689842.2 with a filing date of Nov. 26, 2014. The content of the aforementioned application, including any intervening amendments thereto, is incorporated herein by reference.

### TECHNICAL FIELD

The present invention relates to the technical field of β-sitosterol drugs, more particularly, to a drug sustained release agent based on β-sitosterol and a preparation method thereof.

### BACKGROUND OF THE PRESENT INVENTION

As a plant sterol, the β-sitosterol has distinct effects on reducing serum cholesterol, and it has special significance for functional food to replace cholesterol with β-sitosterol as a liposome material. It is often used for treating type II hyperlipemia and preventing atherosclerosis, and it can also relieve tension of bladder, urethral sphincter and prostate. β-sitosterol is a white crystal powder, odorless and tasteless, melting point 139˜142° C., insoluble in water and soluble in organic solvents.

At present, the existing drugs with β-sitosterol mainly include capsules, tablets and pills, such as Qianshutai β-sitosterol Capsules, Huangbai Capsules, Jiancun Capsules, Relinqing Capsules (with β-sitosterol), anti-inflammatory tablets hemorrhoid, Fukezhidai Tablets, serenoa repens tablets, Erchen Pills etc. But low water solubility of β-sitosterol makes it hard to be absorbed by human bodies, above dosage forms have poor dissolution in gastrointestinal tract, wide fluctuations of plasma concentration and low bioavailability, and generally taking three times a day is needed, so the drug efficacy is significantly restricted.

### SUMMARY OF THE PRESENT INVENTION

The present invention solves existing problems of prior art and problems of β-sitosterol drugs like poor dissolution, wide fluctuations of plasma concentration, low bioavailability by preparing a drug sustained release agent based on β-sitosterol, which has the characteristics of stable drug concentration, high biological activity, good drug solubility and long acting effect.

The content of the present invention is as follows.

A drug sustained release agent based on β-sitosterol is applied to drugs with the β-sitosterol as a main drug component and is prepared from the components including a drug carrier, a hydrophilic gel material, a erodible matrix material and an insoluble matrix material, characterized in that wherein the drug carrier is a β-cyclodextrin-polyamide-amine dendrimer composites, β-sitosterol is from a plant raw material, and a host-guest inclusion complex is composed of the main drug component and the drug carrier according to the mass ratio of 0.1:0.1-0.1:5.

Cyclodextrins are cyclic polysaccharide compounds with 6-12 glucose molecules generated by starch which is under the action of glycosidase of cyclodextrins, the most common cyclodextrins are linked by 6, 7 or 8 glucose molecules through 1,4-glycoside linkage, which is called α-cyclodextrin, β-cyclodextrin and γ-cyclodextrin respectively. As FIG. 1 and FIG. 2 shown, cyclodextrin is a cone-shaped barrel, a cavity with 0.7-1.0 nm diameter is formed in the middle of cyclodextrin, the inner wall thereof with hydrophobic property is composed of hydrogen atom on 3-C and 5-C in glucose molecules and glycoside oxygen atom, while the outer wall thereof with hydrophilicity is composed of 2-C, 3-C and 6-C terminal hydroxyl group. With the peculiar structure, cyclodextrins can use the hydrophobic cavity to cover guest molecule (substances being covered) to form inclusion complex by hydrophobic interaction, hydrogen bonding and Van der Waals' force etc. In three kinds of cyclodextrins, β-C cyclodextrin is easier to combine with most drug molecules due to its proper dimension of cavity.

Polyamide-amine dendrimer presents monodispersion and is a high molecular material, of which the structure and relative molecular mass can be strict controlled, a cavity is formed inside the polyamide-amine dendrimer, end groups can connect with gene, antibody and other bioactive substances by modification, and massive end groups allow each dendrimer to combine more active substances. Compared with previous lipidosome drug carrier, the dendrimer has many advantages, such as stability, no immunogenicity, no toxicity under recommended dosage, and high transport efficiency of bioactive agents.

The β-cyclodextrin is grafted with polyamide-amine dendrimer to form β-cyclodextrin-polyamide-amine dendrimer composites, the detailed preparation reaction thereof is as FIG. 3 shown, the β-cyclodextrin-polyamide-amine dendrimer composites not only has the inclusion function of cyclodextrin for small drug molecules, but also has multiple forms and structural properties of polyamide-amine dendrimer, so drug molecules can be effective covered and controlled release. The β-cyclodextrin-polyamide-amine dendrimer composites is adopted as the drug carrier, so β-sitosterol and other medicinal ingredients from plant can enter into the cavity of β-cyclodextrin hyperbranched polyamide to form stable host-guest inclusion complex, increase the solubility of drugs and relieve the release of drugs.

The preparation method of β-sitosterol inclusion complex includes methods of precipitation, solution, kneading, grinding, ultrasonic wave, freeze drying or spray drying, different methods can be flexibly adopted as needed.

The β-sitosterol plant materials are a mixture of one or more of wormwood, pomelo, Camellia nitidissima Chi, Peristrophe roxburghiana, Arctium lappa L., Verbena officinalis, purslane, Suberect spatholobus stem, Evodia lepta (Spreng.) Merr., mango leaves, Pholidota chinensis lindl, Phyllanthus urinaria L., Thunder god vine, Fructus aurantii, Rubus parvifolius, Hedyotis diffusa, longan, Folium mori, yam, Isatidis radix, Semen litchi, Paederia scandens. Wikstroemia indica, Viola philippica Cav., Patrinia scabiosaefolia fisch, astragalus, Rhizoma cibotii, affine cudweed, phoenix tree flower, Reynoutria sachalinensis, Sarsaparillae radix, Hedychium chrysoleucum Roxb, fistular onion stalk, taxus, ligustrum flower, Rhizoma drynariae, sweet-scented osmanthus, Eucommia ulmoides leaf, maple leaf, root of common fig, cactus, fir bark, alyce clover, hawthorn, radish, carrot, soybean, balsam pear, oat bran, Stigma maydis, grape seed, peanut hull, Rhizoma typhonii, bamboo shoot, Coriaria nepalensis Wall., Pueraria wallichii, rabdosia, Indian kalimeris herb, Uncaria sessilifructus, asparagus fern, notopterygium, Magnolia liliiflora, semen brassicae, fraxinella, pterocypsela laciniata, Stellera chamaejasme L. root, Adenophora wawreana, siberian cocklour fruit, Folium isatidis, leaves of Ligularia veitchiana, Parabarium micranthum (A. DC.) Pierre, Litsea lancifolia, samara oil, Euphorbia latifolia, Peking euphorbia root, Beaumontia grandiflora, Cirsium setosum, Stelmatocrypton khasianum, Flos lonicerae sedum lineare, buckwheat oil, agrimony, celastrus angulatus, scrophulariae, Elaeagnus umbellate, ginseng, Clematis apiifolia DC. var. obtusidentata Rehd. et Wils., Spina gleditsiae, sea buckthorn, Dendrobium fimbriatum, copperleaf herb, Atractylodes macrocephala Koidz., Rhizome typhonii, Radix pseudostellariae, cynomorium, Cistanche salsa, Rhizome pinelliae, Pistacia chinensis, gold lotus, Juglans mandshunrica maxim, pitaya flower, Paris mairei leveille, Rabdosiea Serrae Hare, Herba ecliptae, Zedoary turmeric oil, Alpinia oxyphylla, Chinese rose, Semen cuscutae and Belamcanda chinensis. The materials cost can be reduced effectively due to wide sources.

There are unmodified and modified functional β-cyclodextrin-polyamide-amine dendrimer composites, wherein the modified functional β-cyclodextrin-polyamide-amine dendrimer composites are hydroxyethyl-β-cyclodextrin-polyamide-amine, hydroxylpropyl-β-cyclodextrin-polyamide-amine dendrimer composites, glucosyl-β-cyclodextrin-polyamide-amine dendrimer composites, diglucosyl β-cyclodextrin-polyamide-amine dendrimer composites, carboxymethyl-β-cyclodextrin-polyamide-amine dendrimer composites or sulfobutylether-β-cyclodextrin-polyamide-amine dendrimer composites. Different types of β-cyclodextrin-polyamide-amine dendrimer composites can be flexibly adopted according to requirements of drug efficacy to reach different sustained release effect.

The modified functional polyamide-amine dendrimer include dendrimer from G1.0 to G10.0, different modified functional β-cyclodextrin-polyamide-amine dendrimer composites can further broaden the application range of sustained release drug carrier. With the increase of generations of polyamide-amine, there is hydrogen-bond interaction between cavity of polyamide-amine as well as tertiary amine group of polyamide-amine and drug molecule, the solubility will be distinct increased.

The hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin. The erodible matrix materials are a mixture of one or more of octadecanol, cetyl alcohol, glyceryl behenate, stearic acid, glyceryl monostearate, cholesteryl stearate, camauba wax, hydroxypropyl methylcellulose phthalate, polymethyl methacrylate, triethyl citrate, glyceryl triacetate and stearic acid.

The insoluble matrix materials are a mixture of one or more of acrylic resin, polymethyl methacrylate and ethyl cellulose.

The auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and magnesium carbonate.

The drug sustained release agents can be membrane-controlled release tablets, osmotic pump tablets, matrix tablets, sustained release capsules, sustained release granules or membrane-controlled release pellets. The inclusion complex is mixed with microcrystalline cellulose, polyethylene glycol and other sustained release auxiliaries to prepare sustained release tablets, capsules, granules etc., which can further delay the release of drugs, stabilize plasma concentration and increase the bioavailability of drugs.

A method of the drug sustained release agent based on β-sitosterol, comprising following steps:

The first step: preparing the inclusion complex, the inclusion complex are prepared by β-sitosterol and β-cyclodextrin-polyamide-amine dendrimer composites according to the mass ratio of 0.1:0.1-0.1:5 by adopting methods of precipitation, solution, kneading, grinding, ultrasonic wave, freeze drying or spray drying.

The second step: mixing auxiliaries, β-sitosterol, β-cyclodextrin-polyamide-amine dendrimer composites drug carrier, hydrophilic gel materials, erodible matrix materials, and insoluble matrix materials are weighted respectively according to corresponding technology ratio, and the mixture is mixed sufficiently and evenly.

The third step: carrying out compression moulding, the evenly mixed mixture prepared in the first step is carried out compression moulding by direct compression, granulated compression, pellet compression or coating moulding.

The granulated compression is carried out by dry granulation, wet granulation method or solid phase separation, wherein the coating moulding is carried out by adopting acrylic resin, triethyl citrate, polyethylene glycol, ethyl cellulose or cellulose acetate.

The advantages of the present invention are as follows.

Firstly, stable drug concentration, β-cyclodextrin-polyamide-amine dendrimer composites is used as the drug carrier, meanwhile, the inclusion of small molecule drugs and macromolecular drugs is realized, it is stable released after taking, which is effective to avoid drug concentration fluctuation.

Secondly, high biological activity, cyclodextrins are cyclic polysaccharide compounds with 6-12 glucose molecules generated by starch which is under the action of glycosidase of cyclodextrins, a cavity is formed inside the polyamide-amine dendrimer, end groups can connect with gene, antibody and other bioactive substances by modification, the composites formed by graft of β-cyclodextrin and polyamide-amine dendrimer has many advantages, such as stability, no immunogenicity, no toxicity under recommended dosage, and high transport efficiency of bioactive agents.

Thirdly, good drug solubility, the binding force of cyclodextrins, drug molecules and hydrophobic cavity is hydrophobic interaction, hydrogen bonding and Van der Waals' force, and the binding force of polyamide-amine dendrimer and drug molecules is hydrogen-bond interaction, so it is convenient for drugs to transport after taking and increase the solubility of drugs.

Fourthly, long acting effect, the release process of drug molecule is slow due to a certain volume of hydrophobic cavity inside the cyclodextrin and more end groups of polyamide-amine dendrimer, which is effective to prolong the acting effect.

Fifthly, low cost, it is convenient for mass production due to simple preparation method and wide materials origin of the sustained release agents, so the production cost of drug sustained release agents are reduced.

### BRIEF DESCRIPTION OF THE DRAWINGS

FIG. 1 is a stereochemical structure diagram of cyclodextrin.

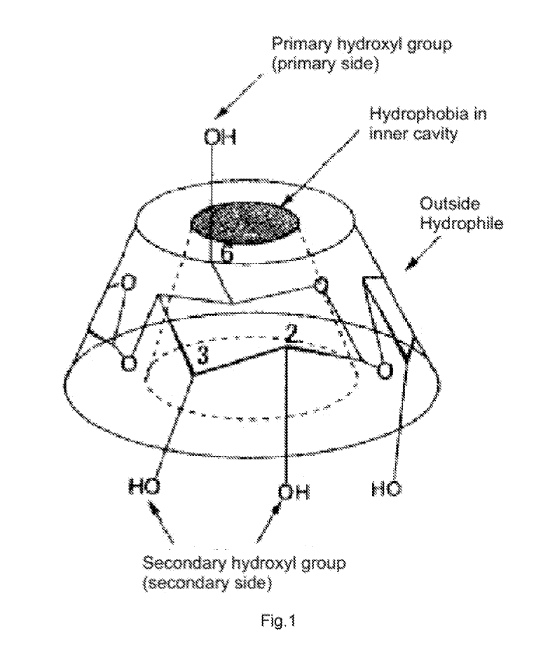

FIG. 2 is a two-dimensional structure of β-cyclodextrin.

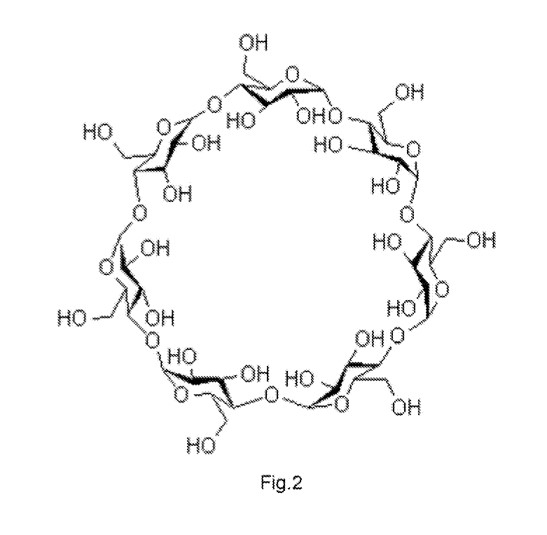

FIG. 3 is a reaction formula of the preparation of β-cyclodextrin-polyamide-amine dendrimer composites.

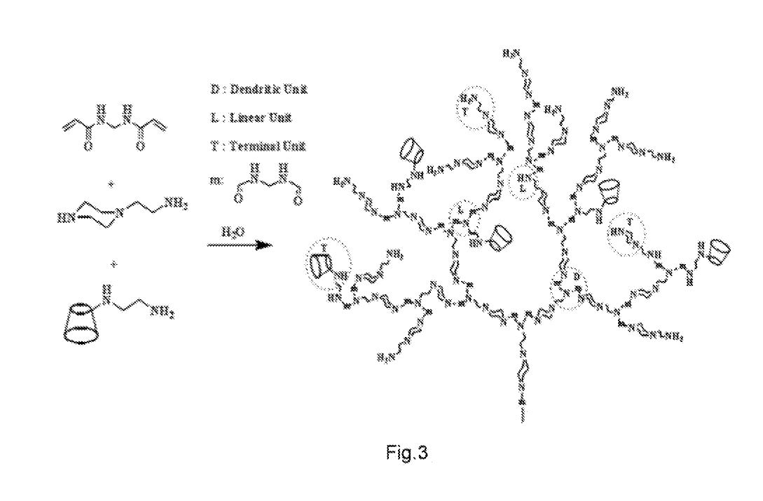

### DETAILED DESCRIPTION OF PREFERRED EMBODIMENTS

In the accompanying drawings. D is dendritic unit, L is the linkage unit, and T is terminal unit.

For better understanding of the present invention, the following is detailed description about summary and embodiments of the present invention.

A method of the drug sustained release agent based on A-sitosterol, comprising following steps:

The first step: preparing the inclusion complex, the inclusion complex is prepared by β-sitosterol and drug carrier according to the mass ratio of 0.1:0.1-0.1:5 by adopting methods of precipitation, solution, kneading, grinding, ultrasonic wave, freeze drying or sprays drying, wherein the drug carrier is β-cyclodextrin-polyamide-amine dendrimer composites.

There are unmodified and modified functional β-cyclodextrin-polyamide-amine dendrimer composites.

The modified functional β-cyclodextrin-polyamide-amine dendrimer composites are hydroxyethyl-β-cyclodextrin-polyamide-amine, hydroxylpropyl-A-cyclodextrin-polyamide-amine dendrimer composites, glucosyl-β-cyclodextrin-polyamide-amine dendrimer composites, diglucosyl β-cyclodextrin-polyamide-amine dendrimer composites, carboxymethyl-β-cyclodextrin-polyamide-amine dendrimer composites or sulfobutylether-β-cyclodextrin-polyamide-amine dendrimer composites.

The amine dendrimer of β-cyclodextrin-polyamide-amine dendrimer composites is unmodified polyamide-amine dendrimer and modified functional polyamide-amine dendrimer, wherein the modified functional polyamide-amine dendrimer include dendrimer from G1.0 to G10.0.

The second step: mixing auxiliaries, β-sitosterol, β-cyclodextrin-polyamide-amine dendrimer composites drug carrier, hydrophilic gel materials, erodible matrix materials, and insoluble matrix materials are weighted respectively according to corresponding technology ratio, and the mixture is mixed sufficiently and evenly.

The hydrophilic gel materials are a mixture of one or more of sodium carboxymethyl cellulose. hydroxypropyl methyl cellulose, calcium alginate, guar gum, chitosan, polyvinyl alcohol, carbopol and DOW polyox water-soluble resin.

The erodible matrix materials are a mixture of one or more of octadecanol, cetyl alcohol, glyceryl behenate, stearic acid, glyceryl monostearate, cholesteryl stearate, camauba wax, hydroxypropyl methylcellulose phthalate, polymethyl methacrylate, triethyl citrate, glyceryl triacetate and stearic acid.

The insoluble matrix materials are a mixture one or more acrylic resin, polymethyl methacrylate and ethyl cellulose. The auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and magnesium carbonate.

The third step: carrying out compression moulding, the evenly mixed mixture prepared in the first step is carried out compression moulding by direct compression, granulated compression, pellet compression or coating moulding.

The drug sustained release agents can be membrane-controlled release tablets, osmotic pump tablets, matrix tablets, sustained release capsules, sustained release granules or membrane-controlled release pellets.

The granulated compression is carried out by dry granulation, wet granulation method or solid phase separation, wherein the coating moulding is carried out by adopting acrylic resin, triethyl citrate, polyethylene glycol, ethyl cellulose or cellulose acetate.

To further explain the effect of drug sustained release agent of present invention, a drug sustained release agent based on β-sitosterol of embodiments 1˜3 is prepared according to the preparation method of the present invention, and the drug release is tested, meanwhile, a release curve is made for the drug sustained release agent of embodiment 1, see table 1 and table 2 for details.

TABLE 1
Main Ratio for Drug Sustained Release
Agent of Different Embodiments

| No. | Main Ingredients | Ratio |
|---|---|---|
| Embodiment 1 | β-sitosterol | 96 g(including 48 g β-sitosterol and |
| | inclusion complex | 48 g β-cyclodextrin-polyamide-amine |
| | | dendrimer) |
| Embodiment 2 | β-sitosterol | 44.6 g(including 6 g β-sitosterol and |
| | inclusion complex | 55 g β-cyclodextrin-polyamide-amine |
| | | dendrimer) |
| Embodiment 3 | β-sitosterol | 44.5 g(including 6 g β-sitosterol and |
| | inclusion complex | 38 g β-cyclodextrin-polyamide-amine |
| | | dendrimer) |

β-sitosterol inclusion complex is prepared by solution method in embodiment 1, grinding method in embodiment 2 and precipitation method in embodiment 3 respectively, mass of which is dosage of preparing 1000 tablets drug sustained release agent.

TABLE 2
Drug Concentration Release Data of Drug
Sustained Release Agent with Time

| | Sampling point | | | | | |
|---|---|---|---|---|---|---|
| No. | 2 h | 4 h | 6 h | 12 h | 18 h | 24 h |
| Embodiment 1 | 28.56% | 44.39% | 52.82% | 79.11% | 89.53% | 99.28% |
| Embodiment 2 | 30.07% | 40.53% | 50.95% | 77.16% | 89.85% | 99.13% |
| Embodiment 3 | 28.69% | 39.77% | 51.29% | 78.16% | 89.81% | 99.35% |

As table two shown, the drug efficacy of drug sustained release agent based on β-sitosterol of the present invention can last 24 hours, which is long duration; the release concentration is first quick and back slow, and the speed slow down gradually with certain concentration gradient, the change of drug concentration is stable.

The above disclosure merely shows several specific embodiments of the present invention, and the present invention is not limited thereto; those ordinary skilled in the art complete the implementation of the present invention without difficulty based on the drawings of description and above disclosure; while it should be noted to those skilled in the art that several variations, modification and improvements can also be made within the scope of technical proposal, and these variations, modification and improvements are equivalent embodiments; moreover, they are also considered within the protective scope of the present invention.

I claim:

1. A drug sustained release agent based on β-sitosterol is applied to drugs with the β-sitosterol as a main drug component and is prepared from the components including a drug carrier, a hydrophilic gel material, a erodible matrix material and an insoluble matrix material, characterized in that the drug carrier is a β-cyclodextrin-polyamide-amine dendrimer composites, β-sitosterol is from a plant raw material, and a host-guest inclusion complex is composed of the main drug component and the drug carrier according to the mass ratio of 0.1:0.1-0.1:5.

2. A drug sustained release agent based on β-sitosterol according to claim 1, characterized in that there are unmodified and modified functional β-cyclodextrin-polyamide-amine dendrimer composites, wherein the modified functional β-cyclodextrin-polyamide-amine dendrimer composites are hydroxyethyl-β-cyclodextrin-polyamide-amine, hydroxylpropyl-β-cyclodextrin-polyamide-amine dendrimer composites, glucosyl-β-cyclodextrin-polyamide-amine dendrimer composites, diglucosyl β-cyclodextrin-polyamide-amine dendrimer composites, carboxymethyl-β-cyclodextrin-polyamide-amine dendrimer composites or sulfobutylether-β-cyclodextrin-polyamide-amine dendrimer composites.

3. A drug sustained release agent based on β-sitosterol according to claim 2, characterized in that the β-sitosterol plant materials are a mixture of one or more of wormwood, pomelo, Camellia nitidissima Chi, Peristrophe roxburghiana, Arctium lappa L., Verbena officinalis, purslane, suberect spatholobus stem, Evodia lepta (Spreng.) Merr., mango leaves, Pholidota chinensis lindl, Phyllanthus urinaria L., Thunder god vine, Fructus aurantii, Rubus parvifolius, Hedyotis diffuse, longan, Folium mori, yam, Isatidis radix, Semen litchi, Paederia scandens, Wikstroemia indica, Viola philippica Cav., Patrinia scabiosaefolia fisch, astragalus, Rhizome cibotii, Affine cudweed, phoenix tree flower, Reynoutria sachalinensis, Sarsaparillae radix, Hedychium chrysoleucum Roxb, fistular onion stalk, taxus, ligustrum flower, Rhizomea drynariae, sweet-scented osmanthus, Eucommia ulmoides leaf, maple leaf, root of common fig, cactus, fir bark, alyce clover, hawthorn, radish, carrot, soybean, balsam pear, oat bran, stigma maydis, grape seed, peanut hull, Rhizome typhonii, bamboo shoot, Conaria nepalensis Wall Pueraria wallichii, rabdosia, Indian kalimeris herb, Uncaria sessilifructus, Asparagus fern, notopterygium, Magnolia liliiflora, semen brassicae, fraxinella, Pterocypsela laciniata, Stellera chamaejasme L. root, Adenophora wawreana, siberian cocklour fruit, Folium isatidis, leaves of Ligularia veitchiana, Parabarium micranthum (A. DC.) Pierre, Litsea lancifolia, samara oil, Euphorbia latifolia, Peking euphorbia root, Beaumontia grandiflora, Cirsium setosum, Stelmatocrypton khasianum, Flos lonicerae, Sedum lineare, buckwheat oil, agrimony, Celastrus angulatus, scrophulariae, Elaeagnus umbellata, ginseng, Clematis apiifolia DC. var. obtusidentata Rehd. et Wils., Spine gleditsiae, sea buckthorn, Dendrobium fimbriatum, copperleaf herb, Atractylodes macrocephala Koidz, Rhizoma typhonii, Radix pseudostellariae, cynomorium, Cistanche salsa, Rhizome pinelliae, Pistacia chinensis, gold lotus, Juglans mandshurica maxim, pitaya flower, Paris mairei leveille, Rabdosiea Serrae Hara, Herba ecliptae, Zedoary turmeric oil, Alpinia oxyphylla, Chinese rose, Semen cuscutae and Belamcanda chinensis.

4. A drug sustained release agent based on β-sitosterol and a preparation method thereof according to claim 2, characterized in that the hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

5. A drug sustained release agent based on β-sitosterol according to claim 2, characterized in that the erodible matrix materials are a mixture of one or more of octadecanol, cetyl alcohol, glyceryl behenate, stearic acid, glyceryl monostearate, cholesteryl stearate, camauba wax, hydroxypropyl methylcellulose phthalate, polymethyl methacrylate, triethyl citrate, glyceryl triacetate and stearic acid.

6. A drug sustained release agent based on β-sitosterol and a preparation method of the drug sustained release agent according to claim 2, characterized in that the insoluble matrix materials are a mixture of one or more of acrylic resin, polymethyl methacrylate and ethyl cellulose.

7. A drug sustained release agent based on β-sitosterol according to claim 4, characterized in that the drug sustained release agents further comprise auxiliary components, wherein the auxiliary components are adhesive, excipient, flavoring agent, filler, wetting agent and/or lubricant, wherein the auxiliary components include a mixture of one or more of lactase, starch, polyvinylpyrrolidone, tween, lauryl sodium sulfate, span, lecithin, urea, sucrose ester, polyoxyethylene aliphatate, polyoxyethylene aliphatic alcohol ether, poloxamer, sodium acid carbonate, sodium carbonate and basic magnesium carbonate.

8. A drug sustained release agent based on β-sitosterol according to claim 5, characterized in that the releasing agents can be membrane-controlled release tablets, osmotic pump tablets, matrix tablets, sustained release capsules, sustained release granules or membrane-controlled release pellets.

9. A preparation method of the drug sustained release agent based on β-sitosterol according to claim 1, characterized in that it comprises following steps:
The first step: preparing the inclusion complex, the inclusion complex is prepared by β-sitosterol and β-cyclodextrin-polyamide-amine dendrimer composites according to the mass ratio of 0.1:0.1-0.1:5 by adopting methods of precipitation, solution, kneading, grinding, ultrasonic wave, freeze drying or sprays drying.
The second step: mixing auxiliaries, β-sitosterol, β-cyclodextrin-polyamide-amine dendrimer composites drug carrier, hydrophilic gel materials, erodible matrix materials, and insoluble matrix materials are weighted respectively according to corresponding technology ratio, and the mixture is mixed sufficiently and evenly.
The third step: carrying out compression moulding, the evenly mixed mixture prepared in the first step is carried out compression moulding by direct compression, granulated compression, pellet compression or coating moulding.

10. A preparation method of the drug sustained release agent based on β-sitosterol according to claim 9, characterized in that the granulated compression is carried out by dry granulation, wet granulation method or solid phase separation, wherein the coating moulding is carried out by adopting acrylic resin, triethyl citrate, polyethylene glycol, ethyl cellulose or cellulose acetate.

11. A drug sustained release agent based on β-sitosterol and a preparation method thereof according to claim 3, characterized in that the hydrophilic gel materials are a mixture of one or more of sodium carboxymethyl cellulose, hydroxypropyl methyl cellulose, calcium alginate, guar gum, chitosan, polyvinyl alcohol, carbopol and DOW polyox water-soluble resin.

12. A drug sustained release agent based on β-sitosterol according to claim 3, characterized in that the erodible matrix materials are a mixture of one or more of octadecanol, cetyl alcohol, glyceryl behenate, stearic acid, glyceryl monostearate, cholesteryl stearate, camauba wax, hydroxypropyl methylcellulose phthalate, polymethyl methacrylate, triethyl citrate, glyceryl triacetate and stearic acid.

13. A drug sustained release agent based on β-sitosterol and a preparation method of the drug sustained release agent according to claim 3, characterized in that the insoluble matrix materials are a mixture of one or more of acrylic resin, polymethyl methacrylate and ethyl cellulose.
